(19) United States
(12) Patent Application Publication
(10) Pub. No.: US 2011/0159080 A1
(43) Pub. Date: Jun. 30, 2011

(54) COMPOSITION COMPRISING LIPOSOME-ENTRAPPED DOXORUBICIN AND METHODS OF ADMINISTRATION

(76) Inventor: Colin Lowery, Bridgewater, NJ (US)

(21) Appl. No.: 12/995,820

(22) PCT Filed: Jun. 3, 2009
(86) PCT No.: PCT/US09/46075

§ 371 Date: Feb. 17, 2011

Related U.S. Application Data
(60) Provisional application No. 61/058,391, filed on Jun. 3, 2008.

(51) Int. Cl. A61K 9/127; A61K 31/704; A61K 39/395; A61P 35/00

(52) U.S. Cl. 424/450; 514/34; 424/133.1

(57) ABSTRACT

Patients suffering from cancer, such as ovarian cancer, may be treated by administering an effective combined amount of a liposomal-entrappeddoxorubicin, an alkylating antineoplastic agent, and a vascular endothelial growth factor inhibitor. In one embodiment, patients suffering from ovarian cancer are treated by administering an effective combined amount of the liposome-entrapped doxorubicin product DOXIL, carboplatin and bevacizumab.

### TECHNICAL FIELD

Methods and pharmaceutical compositions are disclosed for treating cancer by administering an effective combined amount of liposomal-entrapped doxorubicin, an alkylating antineoplastic agent and a vascular endothelial growth factor inhibitor. In one embodiment, methods and pharmaceutical compositions are disclosed for treating ovarian cancer by administering an effective combined amount of polyethyleneglycol-coated liposome entrapped doxorubicin (PLD), such as the PLD product identified by the tradename DOXIL®, carboplatin and bevacizumab.

### BACKGROUND

The following background discussion is provided to facilitate a better appreciation of the technology relating to the inventions disclosed herein. As statements in this discussion may reflect viewpoints of an inventor, they should not be misconstrued as necessarily corresponding to knowledge in the prior art.

The anthracycline antibiotic doxorubicin has a broad spectrum of antineoplastic action and a correspondingly widespread degree of clinical use. In addition to its role in the treatment of breast cancer, doxorubicin is indicated in the treatment of Hodgkin's Disease and non-Hodgkin's lymphoma, hepatocellular and gastric carcinoma, small cell cancer of the lung, soft tissue and bone sarcomas, as well as cancer of the ovary, bladder and thyroid. Unfortunately, toxicity often limits the therapeutic activity of doxorubicin and may preclude adequate dosing.

Liposomes are microscopic lipidic vesicles often having one or more bilayers of a vesicle-forming lipid, such as a phospholipid, and are capable of encapsulating an active drug. Liposomes with an outer surface coating of a hydrophilic polymer are referred to in the art as STEALTH® liposomes. Pegylated liposomal doxorubicin (PLD) (marketed under the tradenames DOXIL and CAELYX) is a doxorubicin formulation in which doxorubicin is encapsulated in liposomes having an outer surface coating of the hydrophilic polymer polyethyleneglycol. More specifically, PLD is manufactured with a lipid having an attached methoxypolyethylene glycol (MPEG) polymer chain, to form liposomes having an outer surface coating of MPEG that serves to protect the liposomes from detection by the mononuclear phagocyte system (MPS) and to increase blood circulation time. PLD was designed to enhance the efficacy and reduce the dose-limiting toxicities of doxorubicin by altering the plasma pharmacokinetics and tissue distribution of the drug. Preclinical results show that PLD prolongs the systemic circulation of doxorubicin, leading to higher concentrations of the drug in tumors and resulting in a reduction in tumor mass and prolonged survival.

The PLD product DOXIL/CAELYX was granted market clearance in 1995 by the US Food and Drug Administration (FDA) for the use in treatment of AIDS-related Kaposi's sarcoma in patients with disease that has progressed on prior combination chemotherapy or in patients who are intolerant to such therapy. In 1996 it was granted market clearance by the European Union's Commission for Proprietary Medicinal Products for the same indication. In 1999, the PLD product DOXIL/CAELYX was granted US market clearance for the use in the treatment of metastatic carcinoma of the ovary in patients with disease that is refractory to paclitaxel- and platinum-based chemotherapy regimens. In January 2003, the European Commission of the European Union has granted centralized marketing authorization to the PLD product DOXIL/CAELYX as monotherapy for metastatic breast cancer in patients who are at increased cardiac risk. In 2007, DOXIL/CAELYX was granted US market clearance in combination with bortezomib for the treatment of patients with multiple myeloma who have not previously received bortezomib and have received at least one prior therapy.

Another antineoplastic agent, carboplatin, is an alkylating antineoplastic agent indicated for the initial treatment of ovarian cancer that has spread beyond the ovaries in established combination with other approved chemotherapeutic agents, and the palliative treatment of patients with ovarian carcinoma recurrent after prior chemotherapy. Other alkylating antineoplastic agents are known, such as cisplatin and oxaliplatin.

Surgery and initial chemotherapy with combination therapy using paclitaxel and carboplatin is the standard of care for first line chemotherapy of advanced epithelial ovarian cancer. While initial response rates are high, long-term survival rates remain disappointing, and efforts continue to develop more effective primary therapy (McGuire, W. P. et al., N Engl J Med, 334(1):1-6 (1996); Ozols, R. F. et al., J Clin Oncol, 21(17):3194-200 (2003)).

After progression or relapse of advanced ovarian cancer, control of symptoms and quality of life are important considerations. The length of the treatment-free interval, defined as the time between the last dose of initial chemotherapy and disease recurrence, is useful for predicting the activity of second-line chemotherapy. Patients relapsing after a treatment-free interval of more than 6 months following the last dose of initial chemotherapy have an overall better prognosis. The longer the treatment-free interval, the more likely second-line chemotherapy treatment will result in an improvement in clinical outcome. After a treatment-free interval of 12 months or greater, patients are usually treated as if they were chemotherapy-naive. The prognosis is worse in patients who relapse within 6 months of the last dose of initial therapy. Therefore, the treatment-free interval (6 months) has become a conventional cut-off period used to categorize the disease at relapse as “platinum-resistant” or “platinum-sensitive”.

Examples of commonly used agents in the setting of relapse are carboplatin-containing regimens, topotecan and the PLD product DOXIL®. Muggia et al. reported a 26% response rate among 35 platinum-refractory subjects treated with DOXIL (Muggia, F. et al. Eur J Cancer, 37 Suppl 9:S15-S18 (2001)). The initial dose was 50 mg/m² every 3 weeks, however, all subjects on study longer than 3 cycles required dose reductions to 40 mg/m² and dose interval delays to 4 weeks. In a larger Phase II study, using a DOXIL dose of 50 mg/m² every 4 weeks, a response rate of 17% was reported (Gordon, A. N. et al., J Clin Oncol, 18(17):3093-100 (2000)). In both studies, progression-free survival was 5-6 months. These results were consistent with studies of salvage treatment with other non-platinum chemotherapies for ovarian cancer, and associated with manageable toxicity and ease of administration.

The phase II data with DOXIL led to a randomized controlled Phase III trial in recurrent ovarian cancer subjects comparing the efficacy of DOXIL with topotecan. Subjects were stratified by platinum sensitivity and presence or absence of bulky disease. DOXIL (50 mg/m²) was given every 4 weeks and topotecan (1.5 mg/m²/day for 5 consecutive days) was prescribed every 3 weeks.

There was no difference in response rates, progression-free survival (PFS) or overall survival (OS) between the two arms. The toxicity profiles of the two drugs were different, with topotecan causing significant myelosuppression and alopecia, whereas DOXIL caused more stomatitis and palmar-plantar erythrodysesthesia (PPE)/hand-foot syndrome (HFS) (Gordon, A. N. et al. Gynecol Oncol, 95(1):1-8 (2004); Gordon, A. N. et al., J Clin Oncol, 19:3312-22 (2001)).

In the subset of subjects with platinum-sensitive disease, the DOXIL-treated group was associated with significantly longer PFS (median PFS: 28.9 vs. 23.3 weeks, p=0.037). In addition, there was a 30% reduction in the risk of death with DOXIL versus topotecan (median OS: 108 vs. 71.1 weeks, p=0.008). Similar trends were reported with longer follow-up.

Re-treatment with carboplatin and paclitaxel is associated with cumulative neurotoxicity (Armstrong, D. K. et al. Oncologist, 7 Suppl 5:20-8 (2002)). When administered in the first-line setting, paclitaxel with carboplatin or cisplatin was associated with grade 1 to 4 neurotoxicity (du Bois, A. et al., J Natl Cancer Inst, 95(17):1320-9 (2003)). Neurotoxicity slowly resolved after therapy discontinuation, but in 20% of patients it persisted for 2 years or longer. Thus, although platinum paclitaxel re-administration provides clinical efficacy for platinum-sensitive ovarian cancer patients, the frequency of clinically significant residual neurotoxicity after first-line treatment underscores the need for an active platinum-based combination therapy that is not associated with this effect.

Because of neurotoxicity concerns associated with taxanes, a randomized Phase III study investigated the benefit of carboplatin versus carboplatin combined with gemcitabine (Pfisterer, J. et al., J Clin Oncol, 24(29):4699-707 (2006)). The study demonstrated a significant improvement in progression free survival and overall response with the gemcitabine-based combination; however, no overall survival benefit was seen. While myelosuppression was significantly more common in the gemcitabine combination, sequelae including neuropathy, febrile neutropenia or infections were uncommon. This study led to the approval of gemcitabine in the platinum-sensitive ovarian cancer setting.

Clinical trials have also explored various dose and schedule combinations of carboplatin and DOXIL in recurrent, platinum-sensitive ovarian cancer. The GINECO phase II trial recruited 105 platinum-sensitive subjects to receive carboplatin (AUC 5) and DOXIL (30 mg/m²) every 4 weeks (Ferrero, J. M. et al., Ann Oncol, 18(2):263-8 (2007)).

The overall response rate was 63%, including 38% complete response, and the median PFS was 9 months. In contrast to the combinations of gemcitabine and carboplatin, hematologic toxicity was minimal, requiring dose delay in only 20% of subjects. This dose and schedule was further analyzed in a Canadian phase II trial in subjects with intermediate-sensitive disease (Stuart, G. et al. Eur J Cancer, 3:272 (2005)). The response rate was 40%, in a patient population with a slightly worse prognosis compared to the GINECO trial. Grade 3-4 adverse events were seen in 10% of subjects.

In another study conducted by the Southwest Oncology Group (SWOG), subjects with recurrent stage III or IV ovarian cancer, a progression-free and platinum-free interval of 6-24 months after first-line platinum-based chemotherapy and up to 12 courses of a non-platinum containing consolidation treatment, were randomized to DOXIL (30 mg/m²) plus intravenous carboplatin (AUC 5) once every 4 weeks (DOXIL arm) or intravenous carboplatin alone (AUC 5) once every 4 weeks (Alberts, D. S. et al., Gynecol Oncol, 108(1):90-4 (2008)). The DOXIL arm enrolled 31 subjects and the carboplatin alone arm enrolled 30, for a total of 61 subjects out of 900 planned. Response rates were 67% for the DOXIL arm and 32% for the carboplatin alone arm (p=0.02). The estimated median PFS was 12 months for the DOXIL arm versus 8 months for the carboplatin alone arm. The estimated median overall survival on the DOXIL arm was 26 months and 18 months on the carboplatin alone arm (p=0.02). Twenty-six percent of the subjects on the DOXIL arm reported grade 4 toxicities, all hematologic in nature. Although the study was closed early because of slow subject accrual following the publication of the results of the ICON4 study the response rate, median PFS and OS results are intriguing (Parmar, M. K. et al., Lancet, 361(9375):2099-106 (2003)). These data again show an advantage to the DOXIL plus carboplatin combination treatment in patients with platinum-sensitive, recurrent ovarian cancer.

Confirmation of the activity of this combination using different doses of DOXIL and carboplatin was also demonstrated by du Bois et al. (duBois, A. et al., Gynecol Oncol, 107(3):518-25 (2007)). In this study, one hundred forty women with recurrent or advanced endometrial (n=31), cervical or vaginal cancer (n=31), uterine sarcomas (n=11), or recurrent platinum-sensitive ovarian cancer (n=67) received six courses of DOXIL 40 mg/m² and carboplatin (AUC 6) every 28 days. In the subset of subjects with ovarian cancer, the response rate was 68%, the median progression free survival was 9.5 months and the median overall survival was 21.4 months. Hematologic toxicities with National Cancer Institute Common Toxicity Criteria (NCI-CTC) grade 3/4 were anemia in 8%, thrombocytopenia in 14%, neutropenia in 24%, and febrile neutropenia in 2% of 652 administered cycles. Grade 3/4 non-hematological toxicities included fatigue (14% of subjects), pain (10%), dyspnea (9%), palmar-plantar erythrodysesthesia (7%), and nausea/vomiting (7%). Dose intensity reached 87.2% for DOXIL and 88.2% for carboplatin. The schedule of carboplatin (AUC 5) and DOXIL (30 mg/m²) every 4 weeks is being taken forward to a large international randomized trial of carboplatin/paclitaxel versus carboplatin/DOXIL for subjects with platinum-sensitive recurrent ovarian cancer.

The importance of angiogenesis in neoplastic development and progression has been established over the past three decades. Tumors adapt and promote the normal angiogenesis process by secreting proangiogenic factors like vascular endothelial growth factor (VEGF) (Weidner, N. et al. N Engl J Med, 324(1):1-8 (1991)).

Vascular endothelial growth factor stimulates vascular growth and increases vascular permeability, which allows tumor growth and expansion. Ferrara, N. et al. Biochem Biophys Res Commun, 333(2):328-35 (2005)). Evidence indicates that increased vascular density within tumors is correlated with decreased disease-free survival in ovarian cancer patients, often independent of known prognostic factors (Alvarez, A. A. et al. Clin Cancer Res, 5(3):587-91 (1999)). Hollingsworth, H. C. et al., Am J Pathol, 147(1):33-41 (1995)). In addition to vessel density, VEGF levels are also highly correlated with disease stage, histologic grade, and outcome in patients with epithelial ovarian cancer (Shen, G. H. et al. Br Cancer, 83(2):196-203 (2000)).

As angiogenesis plays a central role in the physiological function of the healthy ovary, it could therefore be anticipated that abnormal angiogenesis would be especially relevant in ovary cancer. Martin, L. et al., J Clin Oncol, 25(20):2894-901 (2007)). Avastin® (bevacizumab), a vascular endothelial growth factor (VEGF) inhibitor, is a recombinant humanized monoclonal IgG1 antibody that binds to and inhibits the biological activity of human vascular endothelial growth factor (VEGF) in in vitro and in vivo assay systems. AVASTIN, in combination with intravenous 5-fluorouracil-based chemotherapy, is indicated for first- or second-line treatment of patients with metastatic carcinoma of the colon or rectum. AVASTIN is also approved for first-line treatment of patients with unresectable, locally advanced, recurrent of metastatic non-squamous, non-small cell lung cancer. AVASTIN, in combination with paclitaxel, is approved for the first-line treatment of patients with metastatic HER2-negative breast cancer.

Bevacizumab contains human framework regions and the complimentary-determining regions of a murine antibody that binds to VEGF. Bevacizumab is produced in Chinese hamster ovary mammalian cell expression system in a nutrient medium containing the antibiotic gentamicin. Bevacizumab binds VEGF and prevents the interaction of VEGF to its receptors (Flt and KDR) on the surface of endothelial cells. The interaction of VEGF with its receptors leads to endothelial cell proliferation and new blood vessel formation in in vitro models of angiogenesis. Administration of bevacizumab to xenotransplant models of colon cancer in nude (athymic) mice caused reduction in microvascular growth and inhibition of metastatic disease progression. Bevacizumab has shown antitumor activity in a variety of solid tumors including ovarian cancer (Monk, B. J. et al., Gynecol Oncol 96(3):902-5 (2005)). It is approved in combination with intravenous 5-fluorouracil-based chemotherapy for the treatment of metastatic colorectal cancer, in combination with carboplatin and paclitaxel for the treatment of non-small cell lung cancer, and in combination with paclitaxel, for the first-line treatment of patients with metastatic HER2-negative breast cancer. The antibody has shown antitumor activity for a variety of solid tumors, including ovarian cancer.

A number of Phase II trials have investigated the efficacy and safety of bevacizumab single agent or in a combination therapy to treat ovary cancer subjects. A phase II trial of single agent bevacizumab (humanized anti-VEGF monoclonal antibody) in subjects with persistent or recurrent epithelial ovarian cancer was recently reported (Burger, R. A. et al. J Clin Oncol, 25(33):5165-71 (2007)). Eligible subjects had persistent or recurrent epithelial ovarian cancer and had received one to two prior cytotoxic regimens; treatment consisted of bevacizumab 15 mg/kg intravenously every 21 days until disease progression or prohibitive toxicity. Primary endpoints were PFS at 6 months and clinical response. The study consisted of 62 eligible subjects who had received one to two prior cytotoxic regimens. Thirteen subjects (21.0%) experienced clinical response (two complete, 11 partial; median response duration, 10 months), and 25 (40.3%) survived progression free for at least 6 months. Median PFS and overall survival were 4.7 and 17 months, respectively. Grade 3 adverse events at least possibly related to bevacizumab were hematologic (1), GI (3), hypertension (6), thromboembolism (1), allergy (2), hepatic (1), pain (3), coagulation (1), constitutional (1), and dyspnea (1). Grade 4 adverse events included pulmonary embolus (1), vomiting and constipation (1), and proteinuria (1).

In the Genentech-sponsored Phase II trial (AVF 2949), single-agent bevacizumab 15 mg/kg every 21 days was used to treat primary or secondary platinum-resistant subjects. The result showed a response rate of 16% and a 6-month PFS rate of 27% (Cannistra, S. A. et al. J Clin Oncol., 25(33):5180-6 (2007)). The trial was terminated prematurely because of five (11%) GI perforations reported in the first 44 subjects enrolled. Simpkins et al. proposed that the gastrointestinal toxicity was associated with a significant tumor burden involving the bowel in the setting of advanced ovarian carcinoma after failing multiple chemotherapy regimens, as all the five subjects with perforation had radiographic evidence of bowel involvement (Simpkins, F. et al., Gynecol Oncol, 107(1):118-23 (2007)). Furthermore, in their retrospective study that limited bevacizumab treatment to 25 subjects without bowel involvement, there were no cases of perforation in any of these subjects, who were all platinum resistant and had received a median of 5 prior chemotherapy regimens and 3 prior platinum-containing regimens. Nevertheless, abdominal pain, nausea and fever, the typical signs of perforation might warrant particular attention.

Since single agent bevacizumab was well tolerated and its spectrum of toxicity is distinct from that of most cytotoxic drugs, studies of combination therapies as either first-line or second-line treatment in metastatic ovarian cancer are under development (Burger, R. A., J Clin Oncol, 25(20):2902-8 (2007)).

Although progress has been made in the treatment of cancer, there remains a desire for improved therapies.

### SUMMARY

In one aspect, a pharmaceutical composition for treating cancer is provided. The composition comprises an effective combined amount of liposome-entrapped doxorubicin, wherein the liposomes have an exterior coating of hydrophilic polymer chains, an alkylating antineoplastic agent and a vascular endothelial growth factor inhibitor.

In one embodiment, the pharmaceutical composition comprises an effective combined amount of pegylated liposomal doxorubicin (DOXIL), carboplatin and bevacizumab. As will be illustrated herein, a combination of the PLD product DOXIL, carboplatin and bevacizumab yields an advantageous complementary effect useful in treating cancers and/or in slowing the progression of cancers, and/or in increasing the treatment-free interval of cancers, in particular ovarian cancer, fallopian tube cancer and primary peritoneal cancer.

In another aspect, methods of treatment using the pharmaceutical composition are provided. In one embodiment, the method comprises administering to a subject a composition as described above.

In another embodiment, a method of treatment involves a dosage regimen wherein the components of the composition are administered as follows: the PLD product DOXIL and carboplatin are administered to subjects once during a 28 day cycle, and in a preferred embodiment on day 1 of a 28-day cycle; bevacizumab is administered twice during a 28-day cycle, and preferably on days 1 and 15 of a 28-day cycle. In one embodiment, the dosing regimen is repeated every 4 weeks for up to 10 cycles.

In one embodiment, PLD is administered at a dose of (30 mg/m²) and carboplatin is provided in an amount to achieve an area under the curve (AUC) of about 5. In another embodiment, bevacizumab is administered at a dose of 10 mg/kg.

Thus, the compositions and methods are directed to general and preferred embodiments of methods and pharmaceutical compositions for treating or preventing cancers as defined herein.

Additional preferred embodiments, features, and advantages of the compositions and methods will be apparent from the following detailed description.

### DETAILED DESCRIPTION AND PREFERRED EMBODIMENTS

For the sake of brevity, the disclosures of all patents and other publications cited in this specification are incorporated by reference herein.

In one embodiment, a method of treating a subject suffering from or diagnosed with cancer is provided, the method comprising administering to a subject in need of such treatment: an amount of liposome-entrapped doxorubicin, wherein the liposomes have an exterior coating of hydrophilic polymer chains, an amount of an alkylating antineoplastic agent and an amount of a vascular endothelial growth factor inhibitor, the amounts together providing an effective combined amount. In another embodiment, a pharmaceutical composition for treating cancer is provided, comprising: (a) (i) an amount of liposome-entrapped doxorubicin, wherein the liposomes have an exterior coating of hydrophilic polymer chains, (ii) an amount of an alkylating antineoplastic agent, and (iii) an amount of a vascular endothelial growth factor inhibitor, such amounts together providing an effective combined amount; and (b) a pharmaceutically acceptable excipient.

The terms “including”, “containing”, and “comprising” are used herein in their open, non-limiting sense.

The term “subject” refers to a mammalian patient in need of therapeutic or prophylactic treatment. Preferably, subjects treated in accordance with the invention are human.

The term “treat” or “treating” is intended to refer to administration of a composition to a subject for the therapeutic or prophylactic benefit of reversing, ameliorating, alleviating, inhibiting the progress of, lessening the severity of, or preventing a disease, medical condition, or disorder.

The liposome-entrapped doxorubicin is, in a preferred embodiment, comprised of liposomes that have an exterior coating of hydrophilic polymer chains. Such liposome-entrapped doxorubicin products are known in the art, and exemplified by the product known by the tradename DOXIL. This liposome-entrapped doxorubicin has an exterior coating of polyethyleneglycol (PEG), more specifically of methoxypolyethylene glycol (MPEG). The liposome-entrapped doxorubicin for use in the compositions and methods described herein can be widely varied, including but not limited to, the presence or absence of an external surface coating of hydrophilic polymer chains, the polymer forming the hydrophilic polymer chains when present, the lipids forming the liposomal bilayer and the molar ratios of the lipids forming the liposomal bilayer. The various components to form liposomes and techniques for preparation are well known in the art. In a preferred embodiment the liposome-entrapped doxorubicin is comprised of liposomes having an external surface coating of hydrophilic polymer chains, such as polyethyleneglycol, polymethyloxazoline, or polyvinylpyrrolidone. Various components to form liposomes and techniques for preparation are well known in the art.

The alkylating antineoplastic agents of the present invention are alkylating agents that attach an alkyl group to DNA. Alkylating agents work to add alkyl groups to negatively-charged groups such as DNA and are known to stop tumor growth through cross-linking guanine nucleobases in strands of DNA, which directly damages the DNA by making it unable to uncoil and separate. The cell, when attacked in this way, is unable to replicate. While it may not die, it also cannot grow. Preferred alkylating antineoplastic agents for use in the compositions and methods described herein are carboplatin and oxaliplatin.

The vascular endothelial growth factor inhibitors for use in the compositions and methods described herein inhibit angiogenesis. Preferred vascular endothelial growth factor inhibitors are Macugen (Pegaptanib), Lucentis (Ranibizumab) and Bevacizumab (Avastin).

In accordance with the invention, effective amounts of a liposome-entrapped doxorubicin, an alkylating antineoplastic agent and a vascular endothelial growth factor inhibitor are administered to a subject as a treatment regimen for cancer (or its associated symptoms). Cancers that may be treated include, but are not limited to, solid tumors, for example, non-small cell lung cancers, small cell lung cancers, breast cancer, cancer of the pancreas, ovarian cancer, colorectal cancer, prostate cancer, gastric cancer, testicular cancer, bladder cancer, colonic carcinoma, parvocellular and non-parvocellular bronchial carcinoma, carcinomas of the cephalic and cervical parts, carcinomas of the thoracic and abdominal regions, cervical and endometrial carcinomas, sarcomas, melanomas, and leukemias. In one embodiment, cancers that may be treated include ovarian, fallopian tube and primary peritoneal cancers.

Ovarian cancers that may be treated with the compositions and methods described herein include, but are not limited to epithelial tumors that arise from cells that line or cover the ovaries; germ cell tumors that originate from cells that are destined to form eggs within the ovaries; sex cord-stromal cell tumors begin in the connective cells that hold the ovaries together and produce female hormones; and metastatic tumors that begin in other organs but travel to the ovaries. Ovarian tumors are benign, borderline or malignant and some are categorized as advanced.

Fallopian tube cancers that may be treated with the compositions and methods described herein include, but are not limited to primary carcinoma, metastatic carcinomas, adenocarcinomas such as papillary serous, leiomyosarcomas which are tumors that can form from smooth muscle in the fallopian tubes, and transitional cell carcinomas which are tumors from other cells that line the fallopian tubes.

Primary peritoneal cancer (PPC) is a rare cancer that starts in the peritoneum. This is the membrane which lines the inside of the abdomen (tummy), clinging to and covering all the organs in the abdomen (for example the intestines, the liver, and the stomach). This membrane helps to protect the contents of the abdomen. It also produces a lubricating fluid, which helps the organs to move smoothly inside the abdomen as we move around. A primary peritoneal cancer can start in any part of this membrane, usually in the lower part of the abdomen (pelvis).

The term “effective combined amount” means an effective or complementary amount or dose of liposome-entrapped doxorubicin, preferably having an external surface coating of PEG or MPEG, an effective or complementary amount or dose of an alkylating antineoplastic agent and an effective or complementary amount of a vascular endothelial growth factor inhibitor that together are sufficient to generally bring about or effect a desired therapeutic or prophylactic benefit to patients in need of treatment of cancer. The benefit of the effective combined amount of the three agents may be advantageously supra-additive or synergistic, relative to the benefit that would be achieved by administration of the same total amount (i.e., the numerical amount equivalent to the combined effective amount) of one of the agents administered alone, or it may be ameliorative (i.e., the amount of one agent may lessen or reverse an undesirable effect typically induced by administration of the other agents alone).

The term “effective amount” means an amount or dose of liposome-entrapped doxorubicin, preferably having an external surface coating of PEG or MPEG, or an alkylating antineoplastic agent, or a vascular endothelial growth factor inhibitor (as the case may be) that is sufficient to generally bring about or effect a therapeutic or prophylactic benefit when administered alone to patients in need of treatment of cancer.

The term “complementary amount” means an amount or dose of liposome-entrapped doxorubicin, preferably having an external surface coating of PEG or MPEG, or an alkylating antineoplastic agent, or a vascular endothelial growth factor inhibitor (as the case may be) that is: (i) a potentiating amount, which is a dose insufficient to generally bring about or effect the therapeutic or prophylactic benefit when administered alone to patients in need of treatment of cancer (i.e., it is sub-efficacious) but which potentiates or augments the effect of the other agents with which it is administered; and/or (ii) an ameliorative amount, which is a dose of one agent that is sufficient to reverse or lessen the severity of an undesirable side effects generally caused by administration of the other agents alone or in combination.

In certain embodiments, an effective amount of liposome-entrapped doxorubicin, preferably having an external surface coating of PEG or MPEG may be combined with an effective amount of an alkylating antineoplastic agent and a vascular endothelial growth factor inhibitor to provide a therapeutic composition comprising an effective combined amount of the three agents. In preferred embodiments, an effective amount of liposome-entrapped doxorubicin, preferably having an external surface coating of PEG or MPEG is combined with a complementary amount of an alkylating antineoplastic agent which is combined with a complementary amount of a vascular endothelial growth factor inhibitor to provide a therapeutic composition comprising an effective combined amount of the three agents. In other preferred embodiments, an effective amount of an alkylating antineoplastic agent is combined with a complementary amount of liposome-entrapped doxorubicin, preferably having an external surface coating of PEG or MPEG which is combined with a complementary amount of a vascular endothelial growth factor inhibitor to provide a therapeutic composition comprising an effective combined amount of the three agents. It should be understood that the present invention encompasses all of the different orders by which the three agents are combined. In an especially preferred embodiment, an effective amount of a PLD is combined with a complementary amount of caboplatin, which is combined with a complementary amount of bevacizumab.

Suitable amounts of the liposome-entrapped doxorubicin, preferably having an external surface coating of PEG or MPEG, alkylating antineoplastic agent and the vascular endothelial growth factor inhibitor, individually and together, may be ascertained by routine methods, such as modeling, dose escalation studies, or other clinical trials, and by taking into consideration routine factors, e.g., the mode or route of administration or drug delivery, the pharmacokinetics of the agent, the severity and course of the disease, disorder, or condition, the subject's previous or ongoing therapy, the subject's weight, health status, and response to drugs, and the judgment of the treating physician. An exemplary oral dose is in the range of from about 0.001 to about 200 mg of each of liposome-entrapped doxorubicin, alkylating antineoplastic agent and the vascular endothelial growth factor inhibitor per kg of subject's body weight per 24 hours, or preferably about 0.05 to 100 mg/kg/day, or about 1 to 50 mg/kg/day, in single or divided dosage units (e.g., BID, TID, QID). For a 70-kg human, an illustrative range for a dosage amount is from about 0.05 to about 7 g/day, or about 0.2 to about 2.5 g/day, or about 0.01 to about 1 g/day, or about 0.001 to about 0.5 g/day, of each active agent, either in separate dosage forms or in a combined dosage form. Infusion doses can range from about 1 to 1000 μg/kg/min of each agent, admixed with a pharmaceutical carrier over a period ranging from several minutes to several days. For an exemplary topical administration, the active agents may be admixed with a pharmaceutical carrier at a concentration of about 0.1% to about 10% of drug to vehicle. Other suitable dosages for the active ingredients or agents may be routinely determined, e.g., in light of the dosages for liposome-entrapped doxorubicin, alkylating antineoplastic agent and the vascular endothelial growth factor inhibitor exemplified in the art and in investigative and commercial products. For additional guidance, see, e.g., Simons et al., New England J. Medicine, 330:1663-1670 (1994).

Once improvement of the cancer has occurred, the dose may be adjusted for preventative or maintenance treatment, if indicated. For example, the dosage or the frequency of administration, or both, may be reduced as a function of the reduction of intensity or frequency of symptoms, to a level at which the desired therapeutic or prophylactic effect is maintained. Of course, if the medical condition has been alleviated to an appropriate level, treatment may cease. Patients may, however, be intermittently treated on a long-term basis with the combination therapy of the invention upon any recurrence of the medical condition or symptoms thereof.

The combination therapies described herein may be used to treat various cancers, including but not limited to ovarian, fallopian tube and primary peritoneal cancers.

Compounds having alkylating activity or vascular endothelial growth factor inhibitor activity that are known or that become available may be tested for central activity (i.e., to determine if they are centrally acting) by measuring their ability to cause, for example, anti-cancer effects in humans.

The present invention also relates to pharmaceutical compositions for treating cancer, for slowing progression of cancer, and/or for increasing an expected treatment-free interval, comprising: a) an amount of liposome-entrapped doxorubicin, preferably having an external surface coating of a hydrophilic polymer such as PEG or MPEG, an alkylating antineoplastic agent and a vascular endothelial growth factor inhibitor; and b) a pharmaceutically acceptable excipient. In preferred embodiments, the amount of the polymer-coated liposome-entrapped doxorubicin is a potentiating or ameliorative amount. In further preferred embodiments, the amount of the alkylating antineoplastic agent is a potentiating or ameliorative amount. In further preferred embodiments, the amount of the vascular endothelial growth factor inhibitor is a potentiating or ameliorative amount.

Optionally, the polymer-coated liposome-entrapped doxorubicin, an alkylating antineoplastic agent and a vascular endothelial growth factor inhibitor may be combined with additional active ingredients. The additional active ingredients may be co-administered separately with the polymer-coated liposome-entrapped doxorubicin, an alkylating antineoplastic agent and a vascular endothelial growth factor inhibitor of the invention (i.e., each in its own unit dosage form), or one or more of the active ingredients may be delivered together in a single composition or unit dosage form containing the agents in a pharmaceutical composition according to the invention.

In one embodiment, the polymer-coated liposome-entrapped doxorubicin, an alkylating antineoplastic agent and a vascular endothelial growth factor inhibitor are coadministered in separate pharmaceutical formulations. In a preferred embodiment, the two agents are formulated together into a single pharmaceutical composition. In such pharmaceutical compositions, the polymer-coated liposome-entrapped doxorubicin, an alkylating antineoplastic agent and a vascular endothelial growth factor inhibitor, with optional active ingredients, are combined. In a general aspect, a pharmaceutical composition therefore comprises an effective or complementary amount of at least one polymer-coated liposome-entrapped doxorubicin, an effective or complementary amount of at least one alkylating antineoplastic agent and an effective or complementary amount of at least one vascular endothelial growth factor inhibitor, such amounts of agents together providing an effective combined amount; along with a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a pharmaceutical agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the pharmaceutical agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation. Additionally, dosage forms may be prepared as immediate-, timed-, controlled-, or extended-release formulations.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds can be delivered separately or together in the form of tablets or capsules, or as a solution, emulsion, or suspension. Oral tablets may include one or both of the agents and any other active ingredients mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents may also be administered by non-oral routes. For example, the compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent, admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

The combination of polymer-coated liposome-entrapped doxorubicin, an alkylating antineoplastic agent and a vascular endothelial growth factor inhibitor may alternatively be administered by inhalation, via nasal or oral route, e.g., in a spray formulation also containing a suitable carrier.

Certain aspects, features, or advantages of the invention or exemplary or preferred embodiments are illustrated by the following examples.

### EXAMPLES

The following example is illustrative in nature and is in no way intended to be limiting.

### Example 1

### Objectives

The primary objective of the study was to evaluate the objective response rate (complete response (CR)+partial response (PR)) to carboplatin and PLD product (DOXIL) treatment in combination with bevacizumab in subjects with platinum-sensitive recurrent ovarian, fallopian tube or primary peritoneal cancers.

The secondary objectives were to assess the safety profile of carboplatin and the PLD product DOXIL in combination with bevacizumab as well as the following efficacy endpoints: duration of response; progression-free survival (PFS); and time to progression (TTP).

### Overview of Study Design

A single arm, multicenter, open label study was designed to enroll 60 subjects who met all inclusion/exclusion criteria. All subjects received the PLD product DOXIL, carboplatin and bevacizumab by intravenous (IV) infusion for a maximum of ten 28-day cycles. A disease response assessment was done after the completion of Cycles 2, 4, 6, 8 and at the end of treatment. Subjects were followed for six months post-treatment for progression-free survival. Disease progression was defined according to Response Evaluation Criteria in Solid Tumors (RECIST). RECIST is an accepted classification for response to treatment with classification of Complete Response (CR), Partial Response (PR), Progressive Disease (PD) or Stable Disease (SD) (Therasse, P. et al. J Natl Cancer Inst, 92(3):205-16 (2000)).

### Study Population

Subjects with platinum-sensitive recurrent ovarian, fallopian tube and primary peritoneal cancers were enrolled.

### Dosage and Administration

The PLD product DOXIL (30 mg/m²) and carboplatin [area under the curve (AUC) 5] were administered to subjects on day 1 of each 28-day cycle. Bevacizumab (10 mg/kg) was administered on days 1 and 15 of every 28-day cycle. Treatment was repeated every 4 weeks for up to 10 cycles.

| | Carboplatin (AUC | Bevacizumab |
|---|---|---|
| DOXIL (mg/m²) | mg/mL × min) | (mg/kg) |
| Day 1 q28d | Day 1 q28d | Days 1 and 15 |
| 30 | 5 | 10 |

### Efficacy Evaluations/Criteria

The primary efficacy endpoint was the objective response rate to treatment defined as the proportion of subjects achieving a complete response (CR) or a partial response (PR) according to the RECIST criteria.

The secondary efficacy endpoints were duration of response, PFS and TTP. Progression-free survival was defined as the interval between the start of treatment (Cycle 1, Day 1) until disease progression or death from any cause, while TTP was defined as the interval between the start of treatment until disease progression or death due to progression.

Disease progression was defined according to the RECIST criteria.

### Safety Evaluations

Safety was evaluated using adverse events, clinical laboratory tests, and tests for cardiac function after the first 20 subjects were entered and received at least 2 cycles of therapy. Overall safety of the entire cohort was summarized at study completion.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A pharmaceutical composition, comprising:
(a) (i) an amount of a doxorubicin entrapped in liposomes having a surface coating of hydrophilic polymer chains, (ii) an amount of a alkylating antineoplastic agent, and (iii) a vascular endothelial growth factor inhibitor, said amounts together providing an effective combined amount; and
(b) a pharmaceutically acceptable excipient.

2. A pharmaceutical composition according to claim 1, wherein the amount of the doxorubicin-entrapped liposomes is a complementary amount.

3. A pharmaceutical composition according to claim 1, wherein the amount of said alkylating antineoplastic agent is a complementary amount.

4. A pharmaceutical composition according to claim 1, wherein the amount of said vascular endothelial growth factor inhibitor is a complementary amount.

5. A pharmaceutical composition according to claim 1, wherein the doxorubicin-entrapped liposomes have a surface coating of polyethylene glycol hydrophilic polymer chains.

6. A pharmaceutical composition according to claim 5, wherein the polyethylene glycol polymer chains are methoxypolyethylene glycol polymer chains.

7. A pharmaceutical composition according to claim 1, wherein the alkylating antineoplastic agent is carboplatin.

8. A pharmaceutical composition according to claim 1, wherein the vascular endothelial growth factor inhibitor is bevacizumab.

9. A pharmaceutical composition according to claim 1, wherein the doxorubicin entrapped in liposomes is doxorubicin entrapped in liposomes having a surface coating of polyethylene glycol polymer chains, the alkylating antineoplastic agent is carboplatin and the vascular endothelial growth factor inhibitor is bevacizumab.

10. A composition according to claim 1 for treating a subject suffering from or diagnosed with cancer.

11. A composition according to claim 1 for treating ovarian cancer, fallopian tube cancer or primary peritoneal cancer.

12. Use of a composition according to claim 1 for the manufacture of medicament for treating ovarian cancer, fallopian tube cancer or primary peritoneal cancer.

13. Use of a composition according to claim 1 in the preparation of a medicament for treating ovarian cancer, fallopian tube cancer or primary peritoneal cancer, the medicament adapted for administration of the liposome-entrapped doxorubicin once every 28 days, the alkylating antineoplastic agent one every 28 days, and the vascular endothelial growth factor inhibitor twice every 28 days.

14. The use according to claim 13, wherein the medicament is administered for about ten 4 week periods.
